(19) United States
(12) Patent Application Publication
(10) Pub. No.: US 2005/0131299 A1
(43) Pub. Date: Jun. 16, 2005

(54) Differential partial beamforming

(76) Inventors: Brent Robinson, Kirkland, WA (US); Steven R. Freeman, Seattle, WA (US)

Correspondence Address: PHILIPS INTELLECTUAL PROPERTY & STANDARDS, P.O. BOX 3001, BRIARCLIFF MANOR, NY, 10510, US

(21) Appl. No.: 11/006,374

(22) Filed: Dec. 7, 2004

Related U.S. Application Data
(60) Provisional application No. 60/528,554, filed on Dec. 10, 2003.

(51) Int. Cl. A61B 8/06

(52) U.S. Cl. 600/447

(57) ABSTRACT

Each transducer element of a transducer array in an ultrasound imaging system has two substantially identical analog delay circuits, the first of which is supplied with the echo signal received by the transducer element, the second of which is supplied with an inverted version of the echo signal received by the transducer element. After both signals have traversed their respective analog delay paths, one of them is inverted and then both of them are combined in order to substantially suppress the effects of localized common mode signals. Preferably, the first and second analog delay circuits are constructed to be substantially identical.

### CROSS REFERENCE TO RELATED CASES

Applicants claim the benefit of Provisional Application Ser. No. 60/528,554, filed 10 Dec. 2003.

This invention relates to medical ultrasound imaging systems and, in particular, to the reduction of noise in analog delay lines of the pre-beamforming stage of a multiline beamformer.

Ultrasonic imaging systems use ultrasonic acoustic waves to observe the internal organs of subject. The frequency range of the ultrasonic acoustic waves can be described essentially by its lower limit: 20 kHz, roughly the highest frequency a human can hear, although medical ultrasound imaging systems tend to use frequencies in the 2 to 15 MHz range. Ultrasound systems emit ultrasonic pulses which, if not absorbed, echo (i.e., reflect), refract, or are scattered by structures in the body. These echoes, refractions, and back-scatterings are received by the ultrasound system, which translates them into images which can be seen and interpreted by medical personnel.

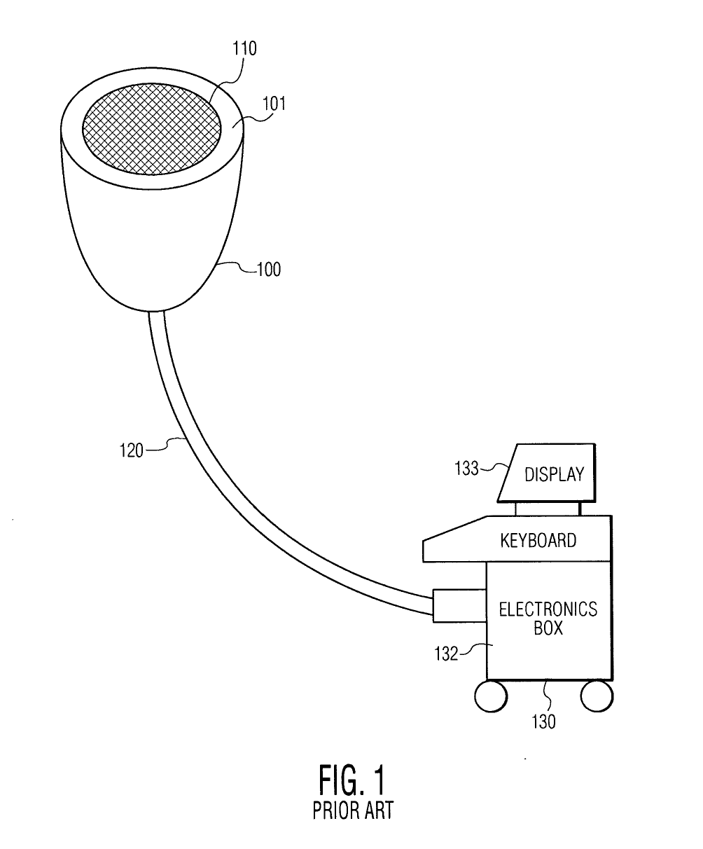

As shown in FIG. 1, a typical ultrasound system is comprised of an ultrasonic transducer (or scanhead) 100, which is typically in the form of a probe which is held by the ultrasound system operator and moved over different portions of the subject's anatomy in order to obtain the desired image. Transducer 100 transmits the ultrasonic waves into the subject, and then receives the resulting signals. Transducer 100 is usually connected to the base ultrasound system 130 by a cable 120, although the transducer can have a wireless connection with the rest of the ultrasound system (e.g., radio transmission; see U.S. Pat. No. 6,142,946 to Hwang et al.). The base ultrasound system 130 comprises processing and control equipment 132, as well as the display 133.

The components which transmit and receive the ultrasonic waves in the transducer may be implemented differently in various ultrasonic systems. In the ultrasound system of FIG. 1, the face 101 of the transducer 100 (which is placed against the flesh of the subject to perform the imaging) consists of an array 110 of piezoelectric elements, which both transmit and receive the ultrasonic waves. In ultrasound systems that use such arrays, the ultrasonic waves are created (and the resulting signals are interpreted) by a process called “beamforming” which is performed mostly in signal processing hardware and software. When transmitting, individual piezoelectric elements in the transducer array 110 are stimulated in particular patterns in order to form and focus one or more ultrasonic beams. When receiving, the signal information received by individual piezoelectric elements in the transducer array 110 are delayed, combined, and otherwise manipulated in order to form electronic representations of one or more ultrasonic beams.

In a “multiline” beamformer, the transducer array 110 transmits a single ultrasonic beam, but the receive beamformer electronics synthesizes several receive ultrasonic beams with different orientations. The oldest and most basic approach to multiline beamforming is to use multiple single line beamformers which are operated in parallel, such as described in U.S. Pat. No. 4,644,795 to Augustine, which is incorporated by reference. In such an arrangement, each element in the transducer array is connected to a channel of the beamformer. Each of these channels applies delays to the signals from its corresponding element which are appropriate to steer and focus the beam being formed by the beamformer. The signals delayed by each channel of the beamformer are combined to form a uniquely steered and focused beam, and the multiple beams produced simultaneously by parallel operated beamformers are used to form multiple lines of an ultrasound image.

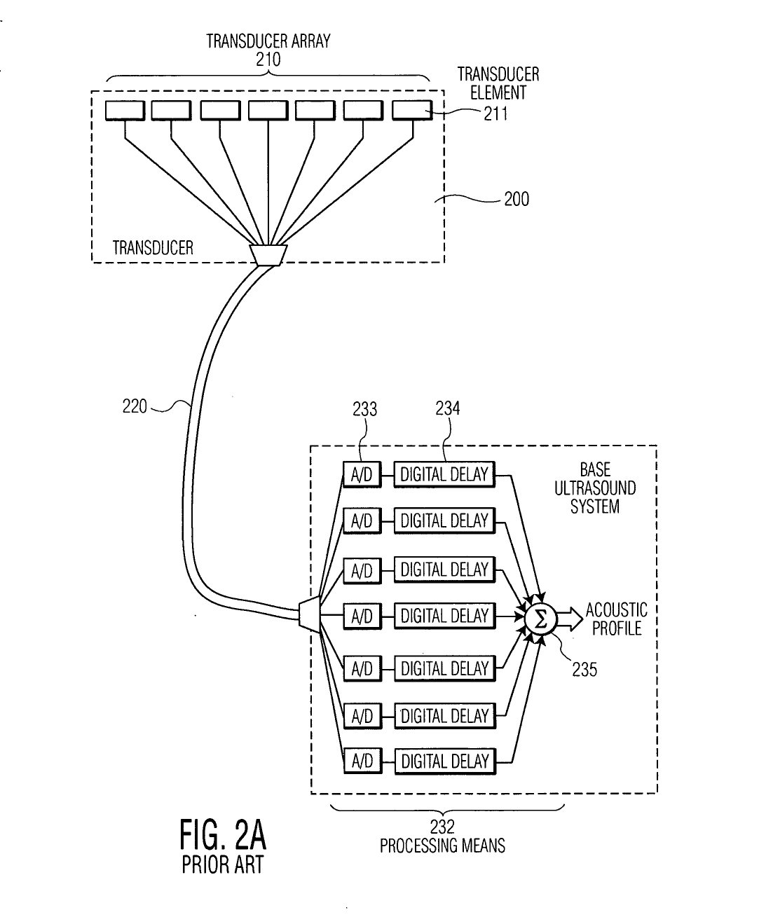

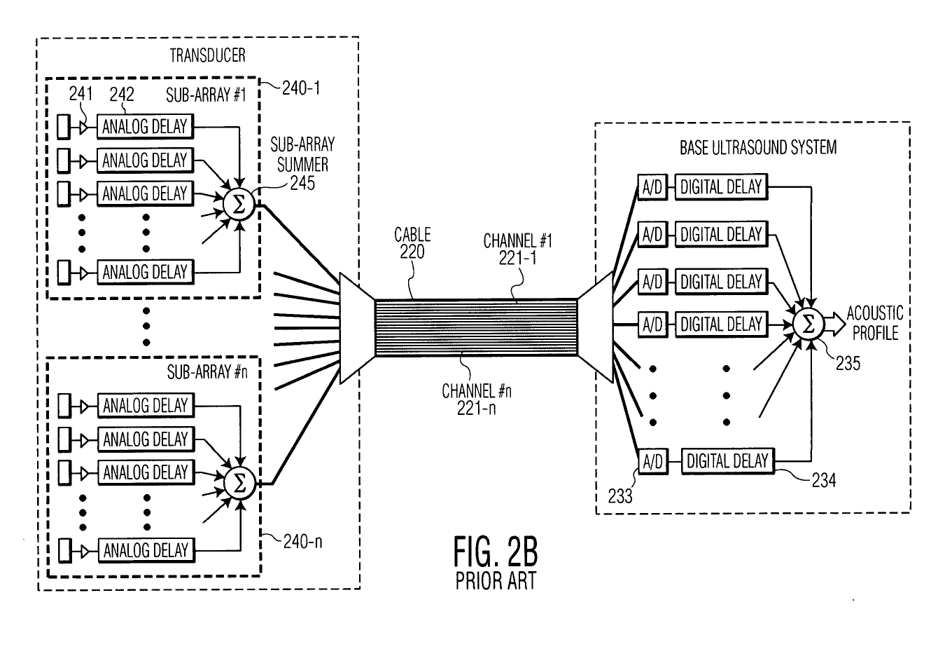

An example of this is shown in FIG. 2A, in which each of the elements 211 of transducer array 210 in transducer 200 has a channel on which its received signals are transmitted over cable 220 to the processing means 232 in the ultrasound system. The signals received by the elements 211 may, or may not, be conditioned in the transducer (e.g., impedance matching) and then transmitted over cable 220 to the ultrasound system. The processing means 232 takes the received signals, which are still in analog form, and converts them to digital signals using analog-to-digital converters (A/D) 233. The resulting digital signals are then delayed by digital delays 234 and summed together by summer 235 to form an acoustic receive sensitivity profile focused at any desired point within the imaging plane.

This approach is sufficient if the number of elements 211 being sampled in the transducer array 210 remain fairly low, i.e., under 200 or so elements (traditional beamformers have 128 channels). However, if the transducer array 210 has thousands of acoustic elements 211, and one desires to use samples from each of those elements, cable 220 would have to carry thousands of channels, which would require a prohibitively large cable and more power than is available from a standard electric outlet (the typical power source for most ultrasound systems). For these and other reasons (including the excessive cost of such a cable and the associated electronics), the approach shown in FIG. 2A is not feasible when fully sampling the ˜3000 elements which may be available in a transducer array.

One solution to this problem of complexity, called “sub-array beamforming”, is shown in FIG. 2B and is described in the paper entitled “Fully Sampled Matrix Transducer for Real Time 3D Ultrasonic Imaging” by Bernard Savord and Rod Solomon (Paper 3J-1, Proceedings of the 2003 IEEE Ultrasonics Symposium, Oct. 5-8, 2003 (IEEE Press)) and U.S. Pat. No. 5,318,033 to Savord, both of which are hereby incorporated by reference. As described therein, and shown in FIG. 2B, in sub-array beamforming, the beamforming function is split into two stages, the first stage taking place in the transducer 200, and the second stage taking place in the processing means 232 of the ultrasound system. By performing partial beamforming in the first stage inside transducer 200, the number of channels required to be transmitted over cable 220 is drastically reduced.

In the example of sub-array beamforming shown in FIG. 2B, individual elements 211 in transducer array 210 are grouped into sub-arrays 240-1 to 240-n. Each element 211 in each sub array 240 has a pre-amplifier 241 and a low power analog delay 242, and each sub-array 240 has a sub-array summer 245 for combining the appropriately delayed signals within the sub-array into one channel. Examples of low power analog delay technology which can be used in the first stage include mixers, phase shifters, charge coupled devices (CCD), analog random access memory (ARAM), sample-and-hold amplifiers, and analog filters. All these technologies have sufficient dynamic range and use sufficiently low power to allow their integration into application-specific integrated circuits (ASICs) capable of fitting inside transducer 200.

When performing beamforming, the signals received by transducer elements at the rim of the array are typically delayed on the order of +/−10 microseconds relative to the signals received by transducer elements in the center of the array. Implementing such a delay in analog delay circuit takes up a lot of room on an integrated circuit; implementing such a delay for hundreds of transducer elements would require too much room, i.e., the ASIC would be too large to fit within transducer 200. In order to keep the aggregate size of these analog delay circuits sufficiently small, it is necessary that each analog delay be implemented in a relatively short length. This required short delay length can be achieved by (1) applying a relative delay to each element, i.e., a delay length which is appropriate relative to the other elements in its sub-array, and (2) having each element within a sub-array located substantially contiguously. Because the relative delays between contiguously located elements are small, the analog delays 242 within each sub-array 240 will be sufficiently small. Preferably, the signals received by the elements within each sub-array 240 will be aligned to within a small fraction of a wavelength at the nominal receive frequency for steering angles of up to 45° or so (neglecting focal delays). After the sub-array signals are formed, different bulk delays may be applied to each sub-array signal, where each bulk delay imposes the appropriate delay on each sub-array relative to the other sub-arrays.

The partially beamformed analog signals from sub-arrays 240-1 to 240-n are transmitted on channels 222-1 to 222-n over cable 220 to processing means 232 in the ultrasound system, where the sub-array analog signals are converted to digital by A/Ds 233, appropriately delayed by digital delays 234, and then combined by final summer 235. The bulk delays discussed in the paragraph above may be implemented by digital delays 234.

Although contiguous, the transducer elements which comprise a sub-array may form a variety of shapes or patterns on the transducer array. For example, in a rectangularly shaped transducer array, each column of transducer elements may form a sub-array. Such a construction is described in U.S. Pat. No. 6,102,863 to Pflugrath et al., which is incorporated by reference, and in U.S. Pat. No. 5,997,479 to Savord et al. In the aforementioned application, “elevation” beamforming (i.e., combining the signals in each column of elements) is performed in the transducer, while “azimuth” beamforming (i.e., combining the row of previously combined columns) is performed by the processing means in the ultrasound system.

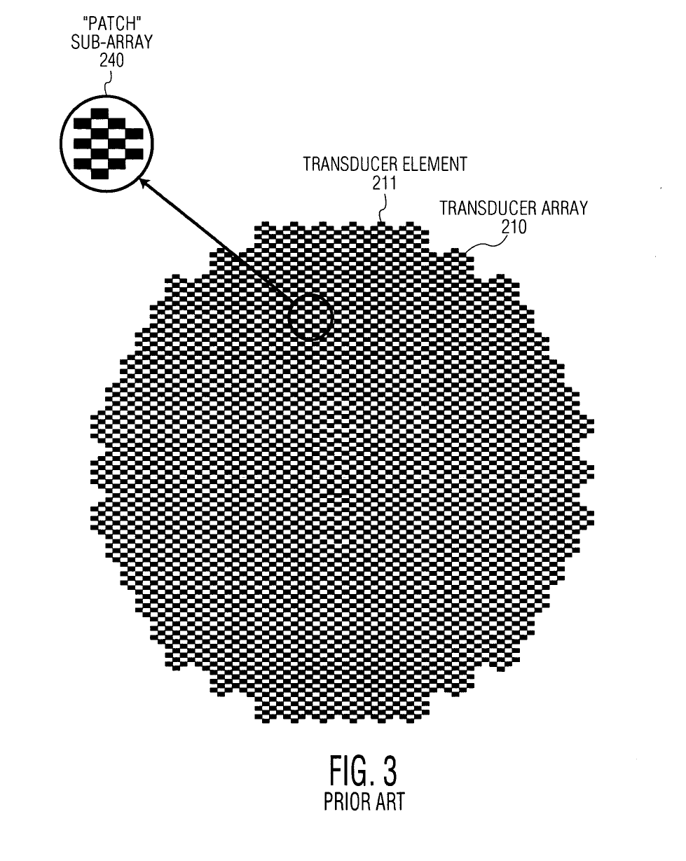

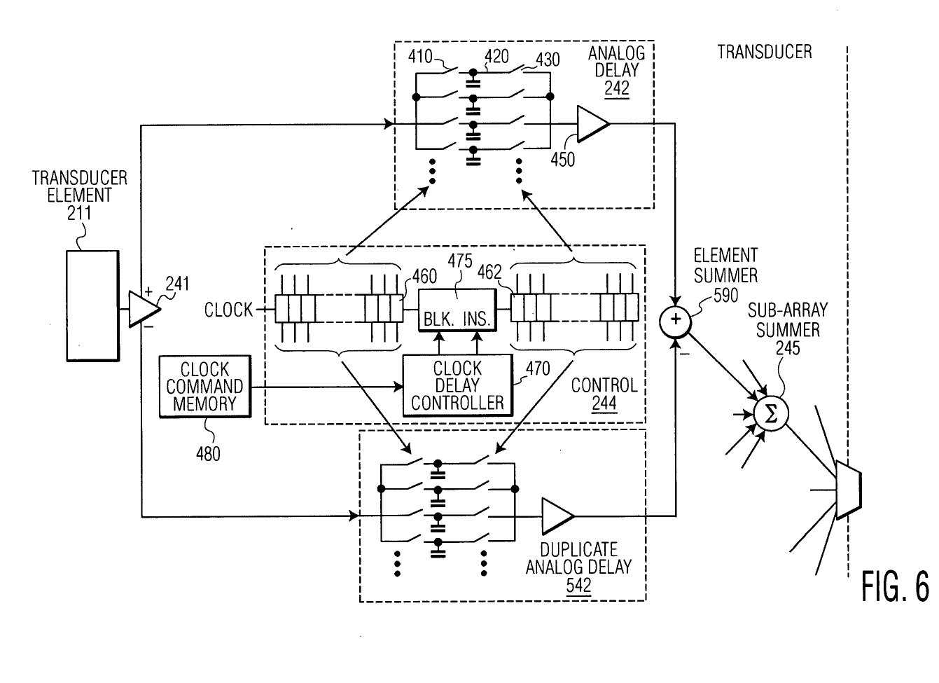

In U.S. Pat. No. 6,682,487 to Savord, filed Sep. 10, 2002, each sub-array forms an irregularly-shaped hexagonal “patch” of twelve transducer elements. As shown in FIG. 3 (a reproduction of FIGS. 6 and 7 of the '291 application), the transducer array 210 is comprised of small boxes each representing a transducer element 211. The overall transducer array 210 has a roughly dodecahedral circumference, in which the sub-array patches are shown as alternating light and dark groupings. One patch 240 is shown circled in the overall transducer array 210, and this patch is shown magnified above and to the left of transducer array 210. Although shown here spaced apart from each other, the transducer elements 211 in an actual patch 240 would be closely packed together in a repeating hexagonal pattern. In the transducer array of the '291 application, the twelve element patch pattern is only used when receiving signals from the subject (i.e., during receive beamforming), whereas a three element pattern is used for transmitting the ultrasonic waves (i.e., during transmit beamforming).

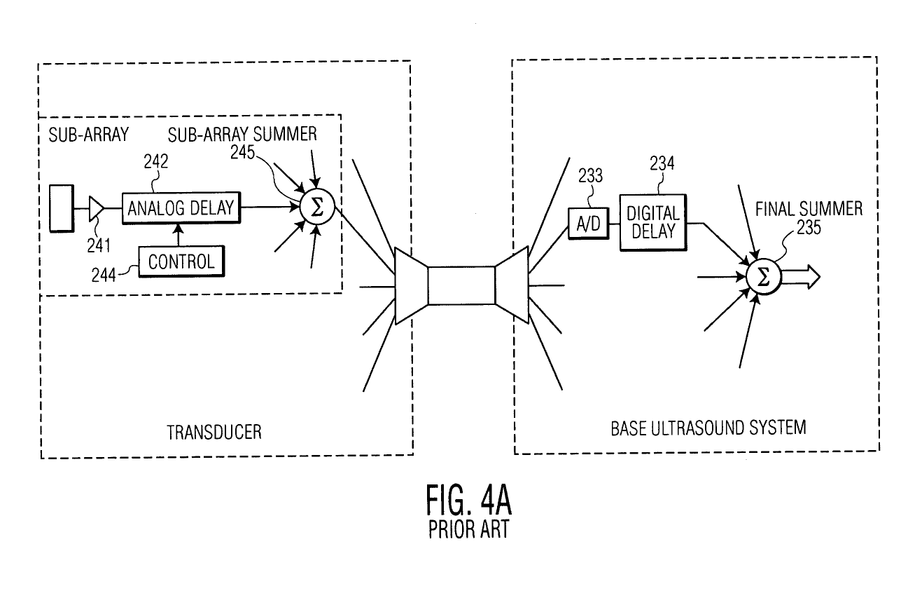

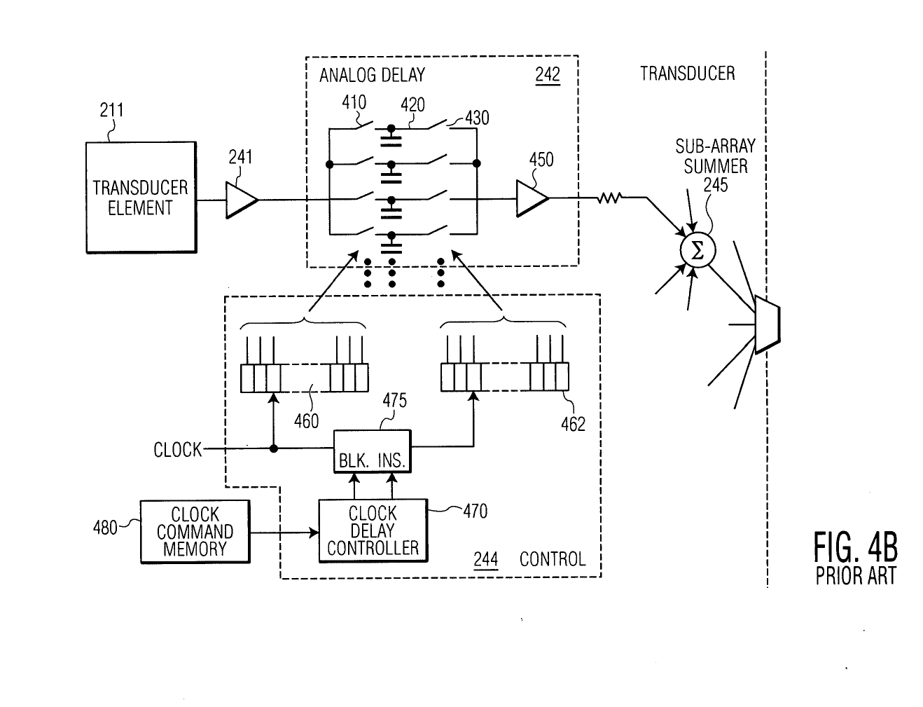

FIG. 4A is a schematic representation of a single analog delay line within a sub-array. As seen in FIG. 4A, the signal received by individual element 211 within sub-array 240 is amplified by pre-amplifier 241 before being appropriately delayed by analog delay 242, which is under the control of control 244. The appropriately delayed signal from analog delay 242 is combined with the appropriately delayed signals from the other elements within sub-array 240 by sub-array summer 245 to form sub-array signal.

FIG. 4B is an exemplary implementation of the single delay line shown in FIG. 4A. As mentioned above, the analog delay may be implemented by any combination of mixers, phase shifters, charge coupled devices (CCD), analog random access memory (ARAM), sample-and-hold amplifiers, and analog filters. The specific implementation shown in FIG. 4B uses analog random access memory (ARAM) to implement analog delay 242. Specifically, the signal received by element 211, after being amplified by pre-amplifier 241, is sampled, i.e., is latched onto one of the capacitors 420. The sampled signal remains stored on the capacitor until it is latched out of the capacitor (thus applying the appropriate delay). The latched out signal is amplified by post-amplifier 450 before being combined with the other signals in the patch sub-array by sub-array summer 245. The timing of latch-in gates 410 and latch-out gates 430 are under the control of two shift registers 460 and 462, respectively, as part of control 244. Each shift register 460-462 is arranged to continually circulate one bit, thereby operating as ring counters. Each bit within a shift register 460-462 is associated with a corresponding gate in the gates 410-430. When the circulating one is shifted into a particular bit in the shift register, the gate corresponding to the particular bit latches (resulting in a signal sample either entering or leaving one of the capacitors 420).

Dynamic receive focus module 475 controls the relative timing between when the signals are sampled by latch-in gates 410 and when sampled signals are fed to sub-array summer 245 by latch-out gates 430 (this is used, for instance, to effect a “focal update”). Dyanamic receive focus module 475 is under the control of clock delay controller 470, which, in turn, is fed control data for forming the current receive beam from clock command memory 480. Although shown here in a particular configuration, dynamic receive focus module 475 can be placed, and/or implemented, in a number of ways.

As can be seen from the exemplary implementation of FIG. 4B, although the signals themselves are stored in analog form, the gates which latch the analog signals are controlled by digital components, such as shift registers 460-462, the clock signal, module 475, clock delay controller 470, etc. Because there are thousands of elements in the transducer array, a high level of integration is required of the components in the patch beamforming ASICs which must fit in the hand-held transducer.

Unfortunately, the close integration of the components in the ASICs brings the digital clock and control circuitry within close proximity of the analog circuitry. Because of this close proximity, digital noise from the various nearby digital components is inevitably coupled onto the analog signals which are either stored on or otherwise acted upon by the analog circuits, and this coupled digital noise reduces the effective dynamic range of the channel. This noise may be coupled capacitively, inductively, or electromagnetically. Because the analog signals have a relatively low voltage level at this stage of the receive signal path, any digital noise coupled to the analog signal can have a considerable effect. Furthermore, because the various analog signals are stored on different capacitors and focal updates occur intermittently, the coupled digital noise is not consistent from signal sample to signal sample and, therefore, the coupled noise does not alias to DC and cannot be completely removed by filtering.

In addition, the high level of integration makes it very difficult to adequately isolate the power and ground lines of the analog circuitry. Digital noise, which is inevitably present on the digital power and ground lines, is usually benign. However, when digital noise contaminates (i.e., is coupled onto) the analog power and ground lines, it is also coupled onto the analog signals being stored and transmitted by the analog circuitry.

In the field of ultrasound imaging, there have been prior art attempts to reduce “common mode signals” (i.e., noise) in general, i.e., noise caused by the general environment, or by large-scale active components in the ultrasound system itself (e.g., the display). For example, in U.S. Pat. No. 4,984,465 to Piel et al., a differential circuit is used to decrease the noise coupled onto the signals being transmitted over the cable from the transducer to the rest of the ultrasound system, where the signals are processed. In the '465 patent, the signals from each transducer element are coupled through a transformer before they are transmitted to the rest of the ultrasound system. These transformers are located in the hand-held transducer. In order to perform the noise reduction, half of the transformers in the '465 patent invert the signal received from its corresponding transducer element, while the other half do not. Thus, the signals on half of the channels transmitted over the cable are inverted, while the signals on the other half are not. Once these signals arrive at the base ultrasound system, the inverted signals are input to the inverted input of the differential circuit, and the non-inverted signals are input into the non-inverted input of the differential circuit. The output of the differential circuit is the sum of the received signals from the transducer elements, with the common mode signals that were coupled to the signals as they were transmitted over the cable suppressed.

In a similar patent from the same inventors, U.S. Pat. No. 5,062,429 to Smith et al., the transducers themselves are polarized in an alternating fashion so that half of the received signals are generated already inverted, thereby suppressing any common mode signals which were coupled anywhere on the receive path from the transducers to the differential amplifier.

However, it would be difficult to implement these prior art solutions on present-day transducer systems. For example, because each element in the transducer in the Piel et al. patent requires a transformer, several thousand transformers would be needed in a typical two-dimensional array transducer, which would not be practical. As another example, the Smith et al. patent requires a complicated connection fixture in order to poll the piezoelectric material after it has been diced into individual array elements, which makes the manufacturing process needlessly complex.

Even if the practical problems were solved, there are more fundamental problems when applying these prior art references to the problem of suppressing coupled digital noise (as described above). To start with, these prior art solutions suppress common mode signals which are affecting multiple transducer elements in the same manner. By contrast, the problem with a single analog delay line, as shown in FIG. 4A, is that each of the signal samples from one single transducer element will be affected differently by the common mode signals. Because each sampled signal is stored on a different capacitor at a different time and each sampled signal travels a different path, the different sampled analog signals will have differing amounts of coupled noise. The prior art solutions will not suppress the time-varying noise effects on the individual delayed signal samples transmitted on a single channel (i.e., received from a single transducer element). Even the noise that is common between multiple channels will not be completely eliminated, because the different delays imparted to the signals on different channels results in the noise on the different channels being not exactly out of phase, and, therefore, the differential amplifier will not completely eliminate the noise.

Furthermore, the prior art solutions are directed to the problem of common mode signals along the entire transmission path from the transducer elements (or transformers) to the ultrasound system itself. By contrast, the problem discussed above in reference to FIGS. 4A and 4B is the noise caused by the tight integration of digital and analog components on an integrated circuit that is contained entirely within the hand-held transducer. In other words, the prior art solutions are directed towards the macro-environment of the ultrasound system, e.g., the effect of electromagnetic noise from the environment surrounding the transmission path of the received signals in the ultrasound system, whereas the problems discussed above concern the micro-environment of densely packed capacitors on an integrated chip in the hand-held transducer.

Therefore, there is a need for a system and method for substantially decreasing the effect of capacitively, inductively, or electromagnetically coupled digital noise, as well as the digital noise coupled via the analog power and ground lines, on the analog signals being processed in the partial beamforming stage within the transducer of an ultrasound imaging system.

One objective of the present invention is to provide a system and method for substantially decreasing the effect of digital noise coupled capacitively, inductively, electromagnetically, or through the analog power and ground lines on the analog signals in the partial beamforming stage in the transducer of an ultrasound imaging system.

Another objective of the present invention is to provide a system and method for decreasing the effect of charge injection on the analog signals in the partial beamforming stage in the transducer of an ultrasound imaging system.

Still another objective of the present invention is to provide a system and method for decreasing the general effect of noise on analog signal samples in an ultrasound imaging system.

These and other objectives are met by the present invention, in which each transducer element has two analog delay circuits, one of which is supplied with the signal received by the transducer element, the other of which is supplied with an inverted version of the signal received by the transducer element. After both signals have traversed their respective analog delay paths, one of them is inverted and then both of them are combined in order to substantially suppress the effects of the localized common mode signals. The combination of the two signals causes any noise which commonly affected both analog delay circuits to be substantially suppressed by cancellation. Preferably, the layouts of the analog delay circuits are substantially the same, and the analog delay circuits are positioned so that they have a substantially similar orientation relative to, and are a substantially similar distance from, other components on the integrated circuit.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

In the drawings:

FIG. 1 shows the large-scale components of a conventional ultrasound imaging system;

FIG. 2A shows a conventional implementation of multiline beamforming in an ultrasound imaging system;

FIG. 2B shows sub-array mulitline beamforming according to the prior art;

FIG. 3 shows an exemplary embodiment of a transducer array with “patch” sub-arrays for multiline beamforming according to the prior art;

FIG. 4A is a schematic representation of a single analog delay line within a sub-array according to the prior art;

FIG. 4B is a specific implementation of the single analog delay line shown in FIG. 4A using analog random access memory (ARAM) according to the prior art;

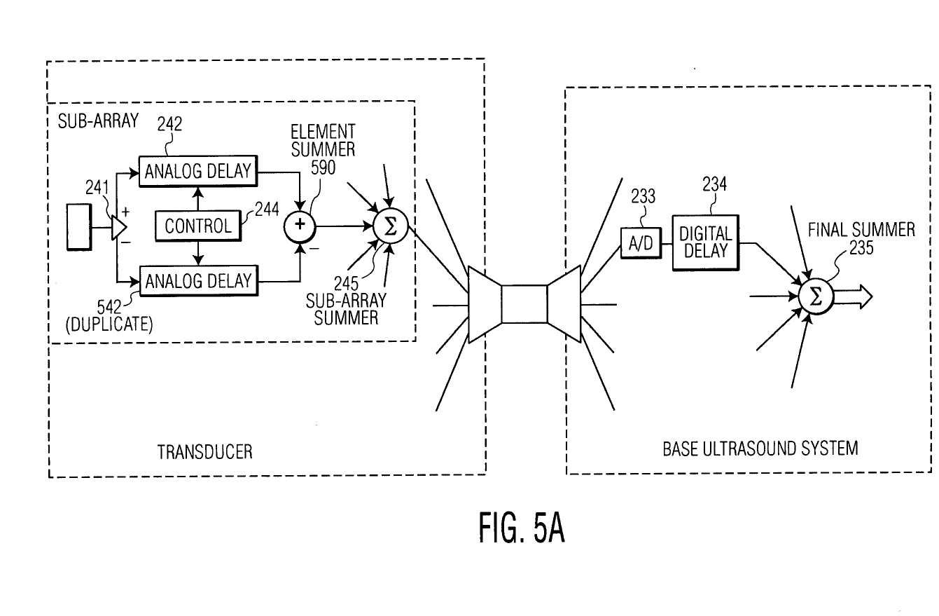

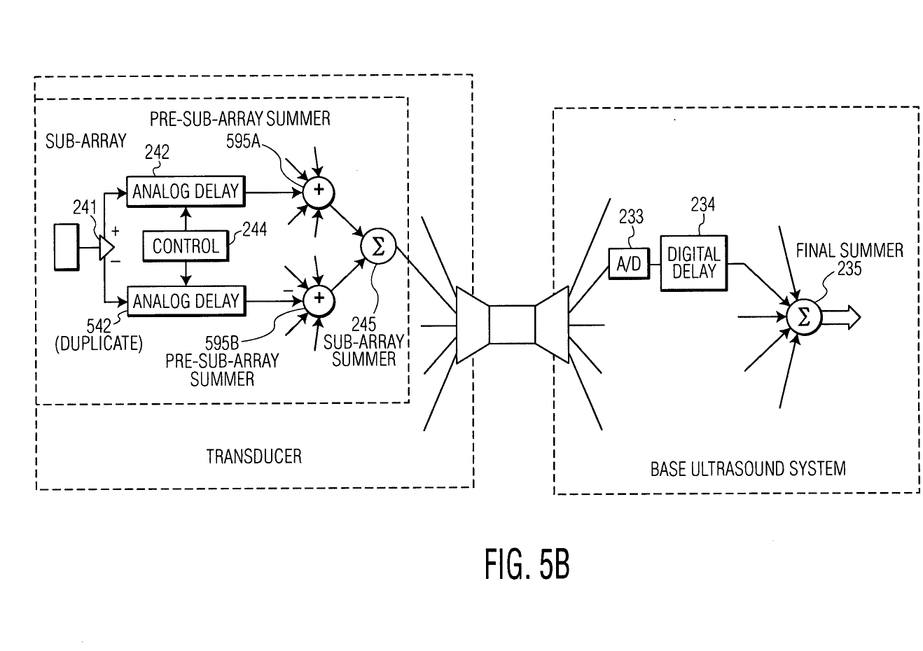

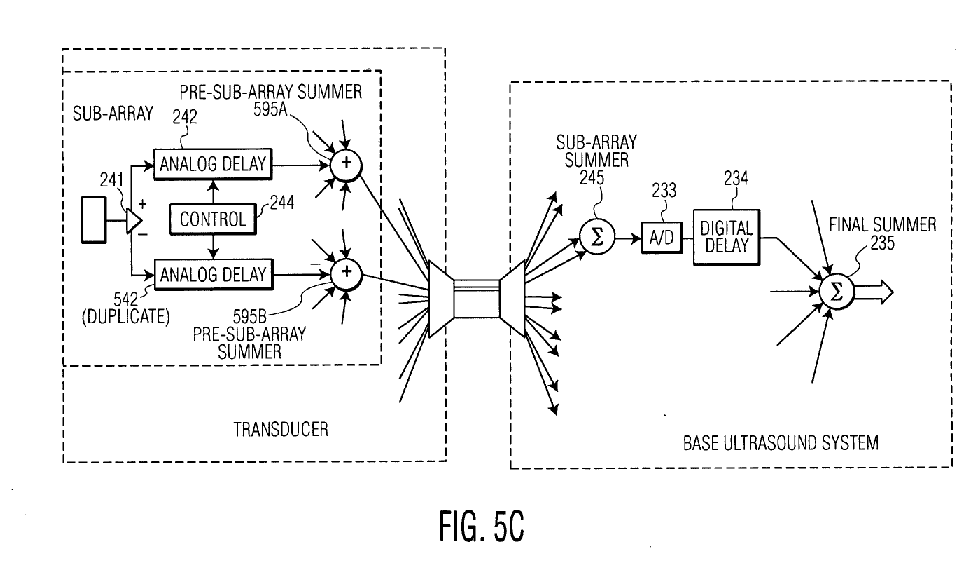

FIG. 5A is a schematic representation of a single analog delay line within a sub-array according to a preferred embodiment of the present invention;

FIG. 5B is a schematic representation of a single analog delay line within a sub-array according to another preferred embodiment of the present invention;

FIG. 5C is a schematic representation of a single analog delay line within a sub-array according to yet another preferred embodiment of the present invention; and

FIG. 6 is a specific implementation of a single analog delay line using analog random access memory (ARAM) according to the preferred embodiment of the present invention shown in FIG. 5A.

The preferred embodiments of the present invention are directed to a system and method for ameliorating the effects of digital noise coupled onto analog signals in the tightly integrated environment of an ASIC used for partial beamforming in an ultrasound imaging system. In general, embodiments of the present invention use duplicate analog delay lines to substantially duplicate, on an inverted copy of the received signal traversing the duplicate analog delay, the effect of localized common mode signals on the original received signal traversing the original analog delay line. After both signals have traversed their respective analog delay paths, one of them is inverted and then both of them are combined in order to substantially suppress the effects of the localized common mode signals.

Because of the two different levels of “partial beamforming” before the “final beamforming” performed in the base ultrasound system, the following nomenclature has been adopted in the claims and in some parts of the specification: the first level of partial beamforming, where the signal from a single transducer element is sampled and appropriately delayed (i.e., by analog delay 242 in FIG. 4A), is called “element beamforming”. The second level of partial beamforming, where the element beamformed signals are combined together to form a sub-array beamformed signal (i.e., by sub-array summer 245), is called “sub-array beamforming”. The final level of beamforming, where the sub-array signals are appropriately delayed and summed together to form the acoustic profile (i.e., by digital delay 234 and final summer 235), may be called “final beamforming” or “beamforming”, as this is the end of the beamforming process.

A schematic representation of a single analog delay line according to a presently preferred embodiment of the present invention is shown in FIG. 5A. The original delay line according to the prior art, comprised of pre-amplifier 241, analog delay 242, sub-array summer 245, and control 244, remains the same, and the combined sub-array signal 247 still travels over cable 220 on channel 222 to the ultrasound system, where it is converted to digital by A/D 233, delayed by digital delay 234, and then finally summed with the other digitally converted and delayed sub-array signals by final summer 235.

However, in contrast to the prior art analog delay line shown in FIG. 4A, the pre-amplifier 241 of the preferred embodiment in FIG. 5A produces two amplified signals, a non-inverted signal which is input into analog delay 242, and an inverted signal which is input to duplicate analog delay 542. Duplicate analog delay 542 is preferably as similar as possible to analog delay 242 in layout, as well as relative orientation to, and distance from, other components (in particular, active components, such as the digital clock and control lines, the analog power and ground lines, and the power and ground planes). Furthermore, the gains on both signal paths should match.

Analog delay 242 and duplicate analog delay 542 both receive the same control signals from control 244 so that the actions of both analog delays are the same. Both output signals (i.e., the non-inverted signal output by analog delay 242 and the inverted signal output by duplicate analog delay 542) are input into element summer 590. However, as indicated by the minus sign next to the inverted signal input into element summer 590, the inverted signal is inverted again when it is input into element summer 590. Element summer 590 combines the non-inverted signal and the twice-inverted signal to output corrected element signal 593 which is input into sub-array summer 245. In other embodiments, the non-inverted signal may be inverted, rather than the inverted signal (for an explanation of why either signal may be inverted, see discussion below). In the preferred embodiment of FIG. 5A, each analog delay line has a duplicate analog delay and an element summer, so that sub-array summer 245 combines corrected element signals from each of the elements in the sub-array.

To explain how the system of FIG. 5A substantially suppresses the common mode signals picked up from digital noise and analog ground and power line noise, we will trace the paths of the non-inverted and twice-inverted signals. First, pre-amplifier 241 outputs a non-inverted signal and an identical, but inverted, signal. When output by pre-amplifier 241, these signals are still fairly pristine representations of the originally received signal. The non-inverted signal has the value +s (represented by voltage), while the inverted signal has the value −s. Second, the non-inverted signal is processed by analog delay 242 while the inverted signal is similarly processed by duplicate analog delay 542. Because both analog delays 242 and 542 have been built as similarly as possible, the effect of noise as the signals are processed is substantially the same on both signals. For purposes of explanation, it will be assumed that the noise coupled through analog delay 542 is identical to the noise coupled through analog delay 242 and, furthermore, that the gains of the two signals paths are identical. Thus, the non-inverted signal is output as +s+n (where n=noise), while the inverted signal is output as −s+n. Third, both delayed signals are input to element summer 590; however, the inverted signal, having a current value of (−s+n), is inverted before being combined with the non-inverted signal, and thus has the value −(−s+n), or (+s−n). When the non-inverted signal and the twice-inverted signal are combined in element summer 590, the result is (+s+n)+(+s−n), or 2s, the noise signal n having been cancelled in the combination.

Even if the noise coupled onto the analog signals in the two analog delays 242 and 542 is not identical, there is still a substantial reduction in noise. Furthermore, the connections to element summer 590 can be reversed so that the non-inverted signal is inverted and the inverted signal is unchanged. In such a case, the output of element summer 590 is −(+s+n)+(−s+n), or −2s. Again, the noise is cancelled. The signal output by element summer 590 is inverted, but this is generally of no consequence for medical ultrasonography as the echo data are passed through a detection stage, which discards the phase, prior to processing the data for display.

The above paragraph is intended as a simplified explanation of the principle by which the present invention substantially reduces common mode signals in the individual analog delay lines of a ultrasound transducer sub-array, and is not intended to limit the invention in any way, shape, or form. For example, the number of times either signal is inverted and the precise location along the receive path for the inversions are not limited (as long as the noise component of one signal serves to substantially cancel the noise component in the other signal when they are combined together). As another example, although the explanation in the above paragraph indicates that the output of element summer 590 is 2s, the output of an element summer in an actual implementation of the preferred embodiment shown in FIG. 5A might be greater or lesser than 2s. As yet another example, because there are now two analog delays where, in the prior art, there was only one, it might be decided to reduce the size of the capacitors in the analog delays 242 and 542 by half in order to conserve space. Although such capacitors would hold only half the charge, when the non-inverted and twice-inverted signals were combined by the element summer 590, the resulting corrected element signal 593 would be back up to full strength (i.e., s). Furthermore, the gain in the summers may be implemented using differential amplifiers, where the gain could differ from unity.

As one can see from the duplicated design elements of the preferred embodiment in FIG. 5A, the analog delay lines of the present invention require greater space and greater power than prior art analog delay lines, such as the one shown in FIG. 4A. The total space and power of the ASIC is not doubled however, because the clock drivers and control circuitry can be shared, and the requirements for the transmit circuitry remain unchanged. As an example, on an ASIC in which the analog delay circuitry takes up about ⅓ of the silicon area, the silicon area required by an embodiment of the present invention would increase the silicon area of the ASIC by a third. However, the increase in performance provided by the substantial diminution in coupled noise in the analog signals may balance the increased size and cost.

FIG. 5B is a schematic representation of a single analog delay line according to another presently preferred embodiment of the present invention. Similarly to FIG. 5A, the prior art delay line, comprised of pre-amplifier 241, analog delay 242, sub-array summer 245, and control 244, remains, and the combined sub-array signal 247 still travels over cable 220 on channel 222 to the ultrasound system, where it is converted to digital by A/D 233, delayed by digital delay 234, and then finally summed with the other digitally converted and delayed sub-array signals by final summer 235. Also similarly to FIG. 5A, the pre-amplifier 241 of the preferred embodiment in FIG. 5B produces two amplified signals, a non-inverted signal which is input into analog delay 242, and an inverted signal which is input to duplicate analog delay 542. Analog delay 242 and duplicate analog delay 542 both receive the same control signals from control 244 so that the actions of both analog delays are the same.

However, in contrast to the preferred embodiment of FIG. 5A, the non-inverted delayed signal output by analog delay 242 and the inverted delayed signal output by duplicate analog delay 542 are not input into a single element summer, but rather input into two separate summers. Specifically, in the preferred embodiment of FIG. 5B, the non-inverted signal output by analog delay 242 is input into pre-sub-array summer 595A, while the inverted delayed signal output by duplicate analog delay 542 is input into the pre-sub-array summer 595B. Pre-sub-array summer 595A also receives the non-inverted signal output from each analog delay 242 within sub-array 240; similarly, pre-sub-array summer 595B also receives the inverted delayed signal output from each duplicate analog delay 542 within sub-array 240. Thus, the output of pre-sub-array summer 595A in FIG. 5B is the same as the output of the sub-array summer 245 of the prior art analog delay line in FIG. 4A. However, the output of pre-sub-array summer 595A is input into sub-array summer 245 of the preferred embodiment of FIG. 5B, as is the output of pre-sub-array summer 595B (inverted, as indicated by the minus sign), and both outputs are combined by sub-array summer 245 to generate sub-array signal 247.

The element summer 590 in FIG. 5A and the sub-array summer 245 in FIGS. 5B and 5C are preferably implemented with differential amplifiers.

As shown by FIGS. 5A and 5B, the location where the non-inverted signal and the twice-inverted signal are combined can be anywhere along the receive beamforming path. In the preferred embodiment of the present invention shown in FIG. 5C, the combination location is moved from inside the transducer downstream to the inside of the ultrasound system.

Similarly to the preferred embodiment shown in FIG. 5B, the preferred embodiment shown in FIG. 5C has two pre-sub-array summers 595A and 595B which combine the non-inverted and inverted signals, respectively of sub-array 240. However, unlike the preferred embodiment shown in FIG. 5B, the sub-array summer 245 is not located within hand-held transducer 200, but is rather located with the processing means 234 in the base ultrasound system 230. Thus, the preferred embodiment in FIG. 5C transmits the two differential signals (output by the two pre-sub-array summers 595A and 595B) from hand-held transducer 200 to base ultrasound system 230 on two separate channels 222A and 222B on cable 220. Because of this, the number of channels carried in cable 220 in FIG. 5C is twice the number of channels carried on cab le 220 in FIG. 5B. Nevertheless, even though it must carry twice the channels, the cable 220 of the preferred embodiment shown in FIG. 5C need not be double the cost of cable 220 in FIG. 5B. The typical prior art cable 220 is a coaxial cable, which is required in order to shield the channels from each other and which is fairly expensive. However, because of the improved noise rejection provided by the differential signaling, the cable 220 in FIG. 5C does not require the shielding provided by coaxial cable, and, thus, could be comprised of much less expensive and easier to terminate cable. For example, cables such as twisted pairs or ribbon cables (e.g., IMAGIN manufactured by W. L. Gore & Associates, Inc.) could be used.

Even though three different preferred embodiments have been shown and described herein, it should be understood that the present invention is not limited to those three preferred embodiments, and embodiments which substitute different elements for some of the elements shown in any and all of the three preferred embodiments are intended to be covered by the scope of the present invention. For example, although all three preferred embodiments described herein use a cable 220 to connect the hand-held transducer 200 to the base ultrasound system 230, an embodiment according to the present invention could replace cable 220 with some form of wireless communication connection between the transducer 200 and base ultrasound system 230. As another example, although all three preferred embodiments described herein have the A/D converters 233, digital delays 234, and final summer 235 located within the base ultrasound system 230, an embodiment according to the present invention could have one or more of these components located outside of the base ultrasound system 230, perhaps in an additional component, or placed within the hand-held transducer 200.

As can be determined from the above description, the present invention provides a system and method for substantially decreasing the effect of digital noise coupled capacitively, inductively, electromagnetically, or through the analog power and ground lines on the analog signals being processed in the sub-array beamforming stage within the transducer of an ultrasound imaging system. However, the present invention also provides a system and method for reducing the effects of other types of common mode signals and, as seen in the preferred embodiment of FIG. 5C, can also be used to ameliorate the effects of common mode signals from the general environment. Furthermore, it is contemplated that other beneficial noise reduction effects are possible with the present invention. For example, there are indications that the present invention may also reduce the charge injection noise of the latch-in and latch-out gates.

As was mentioned above, the analog delay 242 (and duplicate analog delay 542) may be implemented by any combination of mixers, phase shifters, charge coupled devices (CCD), analog random access memory (ARAM), sample-and-hold amplifiers, and analog filters, as would be known to one skilled in the art (and as described in the various references cited in the Background section). In order to provide guidance for the reader, FIG. 6, which shows an exemplary specific implementation of the preferred embodiment in FIG. 5A, is presented herein. Similarly to the specific implementation of a prior art analog delay line shown in FIG. 4B, the exemplary specific implementation of FIG. 6 uses analog random access memory (ARAM) to implement analog delays 242 and 542. Analog delay 242 appears on the top half of FIG. 6, in much the same form as analog delay 242 appears in FIG. 4B. Specifically, there is a bank of capacitors 420, each of which stores a sample of the signal received by element 211 (after being amplified by pre-amplifier 241). A particular capacitor 420 stores the present signal when the latch-in gate 410 of that capacitor closes, thereby allowing the capacitor to store the amplified signal output by the pre-amplifier 241. When the latch-out gate 430 of that particular capacitor 420 closes, the stored (sampled) signal is provided to post-amplifier 450. The time between the latch-in and latch-out of the sampled signal provides the appropriate delay.

Also similarly to FIG. 4A, the set of latch-in gates 410 and the set of latch-out gates 430 are controlled by control 244, and, more particularly, by shift registers 460 and 462, respectively. Shift register 460 controlling the latch-in gates 410 operates as a ring counter under control of the system clock. Shift register 462 controlling the latch-out gates 430 operates as a ring counter under control of module 475. Clock delay controller 470 controls dynamic receive focus module 475, and thereby controls the delay timing of the sampled signals. Clock command memory 480, in turn, controls clock delay controller 470.

However, in contrast to FIG. 4A, the bottom half of FIG. 6 contains duplicate analog delay 542, a close replica of analog delay 242 on the top of the page. The latch-in gates 610 and 630 of duplicate analog delay 542 are also under the control of shift registers 460 and 462, thus the delay timing of the sampled signals in duplicate analog delay 542 should be substantially the same as analog delay 242. Rather than the normal amplified signal, pre-amplifier 241 provides an inverted amplified signal as the input to duplicate analog delay 542. The delayed output of analog delay 242 and the delayed output of duplicate analog delay 542 (after being inverted) are combined by element summer 590 to form corrected element signal 593. The corrected element signal 593 is input into sub-array summer 245. Each analog delay line in sub-array 240 has a duplicate analog delay and an element summer, so that sub-array summer 245 is combining corrected element signals from each of the elements within the sub-array.

As stated above, the specific ARAM implementation shown in FIG. 6 is presented as an example in order to provide general guidance as to how one may implement the preferred embodiment in FIG. 5A, and it is not intended to limit the present invention in any way, shape, or form. The analog delay line according to the present invention may be implemented using any analog technology, including, but not limited to, any combination of mixers, phase shifters, charge coupled devices (CCD), analog random access memory (ARAM), sample-and-hold amplifiers, and analog filters, as would be known to one skilled in the art (and as described in the various references cited in the Background section).

While there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A beamformer for an ultrasound imaging system, said ultrasound system having a array of transducer elements that define a plurality of sub-arrays, comprising:
receive circuitry forming an analog beamforming stage, said receive circuitry responsive to transducer signals generated by receive elements in the transducer array, and wherein, for at least one element in a sub-array, said receive circuitry comprises:
first analog delay circuitry for receiving a non-inverted analog signal from said at least one element, for delaying the non-inverted signal in order to perform beamforming, and for outputting the delayed non-inverted signal at specified times in order to output a non-inverted element beamformed signal;
second analog delay circuitry for receiving an inverted analog signal from said at least one element, for delaying the inverted signal in order to perform beamforming, and for outputting the delayed inverted signal at specified times in order to output an inverted element beamformed signal; and
digital control circuitry for controlling the pair of first and second analog delay circuits such that the delaying of signals and the specified times for outputting are substantially the same;
wherein the signals processed by the pair of first and second analog delay circuits remain in analog form; and
wherein said second analog delay circuitry is constructed so that at least one of (i) its layout is substantially identical to the layout of the first analog delay circuitry, and (ii) the noise coupled on the analog signals processed therein is substantially the same as the noise coupled on the analog signals processed in said first analog delay circuitry;
wherein at least one of the inverted element beamformed signal and the non-inverted element beamformed signal is inverted, wherein, after said inversion, both element beamformed signals are combined, and wherein said combining reduces coupled noise from the resulting combination.

2. The beamformer of claim 1, wherein the coupled noise is coupled at least one of capacitively, inductively, or electromagnetically.

3. The beamformer of claim 1, wherein the coupled noise reduced by combining the element beamformed signals was caused by at least one of digital circuitry, analog power lines, analog ground lines, and charge injection.

4. The beamformer of claim 3, wherein the digital circuitry causing the coupled noise comprises at least one of the digital control circuitry in the receive circuitry and a clock signal.

5. The beamformer of claim 1, wherein the second analog delay circuitry is constructed so that the noise coupled on the analog signals processed therein is substantially the same as the noise coupled on the analog signals processed in said first analog delay circuitry, and wherein the construction of the second analog delay circuitry is substantially the same as the construction of the first analog delay circuitry in at least one of: layout, distance from one or more digital circuits, distance from at least one analog ground line, and distance from at least one analog power line.

6. The beamformer of claim 1, wherein, for at least one sub-array in the array, said receive circuitry further comprises:
a sub-array summation circuit for outputting a sub-array beamformed signal.

7. The beamformer of claim 6, wherein, for each element in said at least one sub-array, said receive circuitry further comprises:
an element summation circuit for receiving the non-inverted element beamformed signal and the inverted element beamformed signal, for inverting at least one of the inverted element beamformed signal and the non-inverted element beamformed signal, and, thereafter, for summing both element beamformed signals in order to produce a corrected element beamformed signal;
wherein the sub-array summation circuit receives the corrected element beamformed signals from each element summation circuit in said at least one sub-array, sums the corrected element beamformed signals, and outputs said sum as the sub-array beamformed signal.

8. The beamformer of claim 6, wherein, for at least one sub-array in the array, said receive circuitry further comprises:
a first pre-sub-array summation circuit for receiving non-inverted element beamformed signals from each first analog delay circuit in said at least one sub-array, for summing the non-inverted element beamformed signals, and for outputting the sum of the non-inverted element beamformed signals; and
a second pre-sub-array summation circuit for receiving inverted element beamformed signals from each second analog delay circuit in said at least one sub-array, for summing the inverted element beamformed signals, and for outputting the sum of the inverted element beamformed signals;
wherein the sub-array summation circuit receives the output sum of the non-inverted element beamformed signals and the output sum of the inverted element beamformed signals, inverts one of the output sums, thereafter sums the output sums, and outputs said sum as the sub-array beamformed signal.

9. The beamformer of claim 6, further comprising:
receive circuitry forming a digital beamforming stage, said receive circuitry for receiving the sub-array beamformed signals from a plurality of sub-array summation circuits, said receive circuitry comprising:
a plurality of analog-to-digital converters for converting received sub-array beamformed signals and for outputting digitized sub-array beamformed signals;
a plurality of digital delay circuits for delaying the digitized sub-array beamformed signals and for outputting delayed and digitized sub-array beamformed signals; and
a final summation circuit for summing the delayed and digitized sub-array beamformed signals and for outputting a final receive beamformed signal.

10. The beamformer of claim 9, wherein the receive circuitry forming the analog beamforming stage is located in a transducer of the ultrasound imaging system, wherein the sub-array beamformed signals are transmitted from the transducer to a base processing means of the ultrasound system, and wherein the receive circuitry forming the digital beamforming stage is located in the base processing means of the ultrasound imaging system.

11. The beamformer of claim 10, wherein the sub-array beamformed signals are transmitted using electromagnetic energy which is transmitted at least one of over a cable and through the air.

12. The beamformer of claim 6, wherein the sub-array summation circuit comprises a differential amplifier.

13. The beamformer of claim 7, wherein the element summation circuit comprises a differential amplifier.

14. The beamformer of claim 1, wherein a means by which at least one of the inverted element beamformed signal and the non-inverted element beamformed signal is inverted, and then both element beamformed signals are combined comprises a differential amplifier.

15. The beamformer of claim 1, wherein, for at least one element in a sub-array, said receive circuitry further comprises:
a pre-amplifier for receiving a signal from the element, for amplifying the received signal, and for outputting the non-inverted analog signal for the first analog delay circuit and the inverted analog signal for the second analog delay circuit.

16. The beamformer of claim 1, wherein the receive circuitry forming the analog beamforming stage is located in a transducer of the ultrasound imaging system.

17. The beamformer of claim 1, wherein the receive circuitry forming the analog beamforming stage comprises an application-specific integrated circuit (ASIC).

18. The beamformer of claim 1, wherein the pair of first and second analog delay circuits comprises at least one of a mixer, a phase shifter, a charge coupled device (CCD), an analog random access memory (ARAM), a sample-and-hold amplifier, and an analog filter.

19. The beamformer of claim 1, wherein each sub-array of the plural sub-arrays comprises a group of contiguous transducer elements in the array.

20. The beamformer of claim 6, wherein each sub-array of the plural sub-arrays comprises a line of contiguous transducer elements, a regularly-shaped polygon of contiguous transducer elements, or an irregularly shaped polygon of contiguous transducer elements.
